(19) United States
(12) Patent Application Publication
(10) Pub. No.: US 2006/0252142 A1
(43) Pub. Date: Nov. 9, 2006

(54) Method for nucleic acid isolation and an instrument for nucleic acid isolation

(75) Inventors: Yoshihiro Yamashita, Hitachinaka (JP); Toshinari Sakurai, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH-TECHNOLOGIES CORPORATION

(21) Appl. No.: 11/418,031

(22) Filed: May 5, 2006

(30) Foreign Application Priority Data
May 6, 2005 (JP) 2005-134728

(51) Int. Cl. C12N 1/08; C07H 21/04

(52) U.S. Cl. 435/270; 536/25.4

(57) ABSTRACT

It is an objective of the present invention to isolate RNA from a sample containing nucleic acid by safe and convenient operations. As a result of intensive studies, inventors of the present invention have found that DNA is precipitated out by adding an organic solvent to a mixed solution of a sample containing DNA and RNA and a chaotropic agent, so that RNA remains soluble. The present invention relates to a method whereby a sample containing nucleic acid, a chaotropic agent, and an organic solvent are mixed, DNA is precipitated out, and the precipitate is separated from the mixed solution, such that RNA is isolated from the residual solution. In addition, in accordance with the present invention, RNA is allowed to come into contact with a silica-containing solid phase so as to be bound to the silica-containing solid phase without the addition of a reagent or the like to the residual solution. Further, it is also possible to isolate DNA from the precipitate. In accordance with the present invention, high-purity RNA can be isolated from a sample containing DNA and RNA by safe and convenient operations. In addition, it is possible to simultaneously isolate RNA and DNA from a single sample.

### BACKGROUND OF THE INVENTION

1. Field of the Invention

The present invention relates to a technique for nucleic acid isolation from a sample containing nucleic acid. For instance, the present invention relates to a technique for RNA isolation from a biological sample containing DNA and RNA.

2. Background Art

DNA (deoxyribonucleic acid) and RNA (ribonucleic acid) are substances responsible for genetic information in organisms. In general, DNA is a substance responsible for all of the genetic information of organisms. Meanwhile, RNA is a substance responsible for protein synthesis in vivo based on the genetic information contained in DNA. Mainly, gene sequence information can be obtained based on DNA analysis, and gene expression information can be obtained based on RNA analysis. These are highly important forms of molecular biological information. When conducting DNA analysis and RNA analysis, in general, pretreatment operation to isolate DNA and RNA from a sample such as a biological sample containing DNA and RNA is essential. During DNA and RNA isolation, it is necessary to avoid contamination with an inhibitor of PCR or RT-PCR analysis, for example. In particular, upon RNA isolation, it is important to avoid contamination with DNA that can be an inhibitor of RNA analysis. In addition, it is required to isolate high-purity RNA. Further, when efficiently conducting DNA and RNA analysis, it is desired that DNA and RNA be simultaneously isolated from a single sample.

An example of a method for RNA isolation from a biological sample is described in Analytical Biochemistry 162, 156-159 (1989). This method comprises the following steps: (1) a biological sample is lysed in guanidine thiocyanate solution, and an acidic buffer solution, a phenol solution, and a chloroform solution are added thereto in that order, followed by mixing; (2) the resulting solution is separated into an aqueous phase containing RNA, an organic solvent phase containing denatured protein and separated DNA, and an aqueous intermediate phase by centrifugation; (3) ethanol or isopropanol is added to the aqueous phase containing RNA; and (4) separated RNA is selectively precipitated by centrifugation. Compared with a conventional method for ultracentrifugation, this method is advantageous in terms of efficient RNA isolation; however, highly hazardous phenol and chloroform must be used in the method. In addition, DNA and RNA cannot be simultaneously isolated from a single sample.

An example of a method for nucleic acid isolation whereby phenol, chloroform, and the like are not used and there is no need to conduct an operation such as ethanol precipitation or isopropanol precipitation is a method whereby the binding characteristics of nucleic acid with respect to a silica-containing solid phase under the presence of a chaotropic agent is utilized (described in Proc. Natl. Acad. Sci. USA, B. Vogelstein and D. Gillespie, 76(2), 615-619 (1979) and J. Clin. Microbiol. 28(3), R. Boom et al., 495-503 (1990)). However, RNA isolated by such method contains DNA. In addition, DNA and RNA cannot be simultaneously isolated via this method.

A method described in JP Patent Publication (Kohyo) No. 2002-507121 A is an example of a method whereby a biological sample is lysed in a chaotropic agent; an aqueous solution containing salts, a buffer agent, and the like is added to a lysis reagent; to precipitate out contaminants containing DNA; and RNA can be extracted from the resulting solution from which a precipitate has been removed. With this method, it is necessary to reestablish the binding condition of RNA with respect to a silica-containing solid phase by adding a solution such as alcohol after removing the precipitate, resulting in complicated operations for isolation. In addition, DNA and RNA cannot be simultaneously isolated from a single sample.

A method described in JP Patent Publication (Kokai) No. 2002-187897 A is an example of a method whereby the binding characteristic of nucleic acid with respect to a silica-containing solid phase under the presence of a chaotropic agent is applied to DNA and RNA isolation. In the case of RNA isolation according to this method, RNA selectivity is insufficient, and thus the resultant contains DNA. In addition, in order to simultaneously isolate RNA and DNA from a single sample, it is necessary to separately establish the binding condition of DNA with respect to a silica-containing solid phase and the binding condition of RNA with respect to a silica-containing solid phase, resulting in complicated operations for isolation.

An example of a method whereby DNA and RNA can be simultaneously isolated from a single sample is described in Molecular Cloning Third edition 7.9 as “A Single-step Method for the Simultaneous Preparation of DNA, RNA, Protein from Cell and Tissue.” This method comprises the following steps: (1) a biological sample is lysed in a lysis reagent containing phenol, guanidine thiocyanate, or the like; (2) chloroform is mixed therein; (3) the resulting solution is separated into an aqueous phase containing RNA and an organic phase containing DNA and protein by centrifugation; (4) RNA is isolated from the aqueous phase by isopropanol precipitation; and (5) DNA is isolated from the organic phase by ethanol precipitation. In accordance with this method, DNA and RNA can be simultaneously isolated from a single sample; however, highly hazardous phenol and chloroform are used, and complicated operations such as operations for preparative isolation of the aqueous phase and the organic phase and multiple operations for centrifugation are required.
[Patent Document 1] JP Patent Publication (Kohyo) No. 2002-507121 A
[Patent Document 2] JP Patent Publication (Kokai) No. 2002-187897 A
[Non-Patent Document 1] Analytical Biochemistry 162, 156-159 (1989)
[Non-Patent Document 2] B. Vogelstein and D. Gillespie, Proc. Natl. Acad. Sci. USA, 76(2), 615-619 (1979)
[Non-Patent Document 3] R. Boom et al., J. Clin. Microbiol. 28(3), 495-503 (1990)
[Non-Patent Document 4] Molecular Cloning Third edition 7.9, A Single-step Method for the Simultaneous Preparation of DNA, RNA, Protein from Cell and Tissue

### SUMMARY OF THE INVENTION

It is an objective of the present invention to isolate RNA from a sample containing nucleic acid via safe and convenient operations.

As a result of intensive studies, inventors of the present invention have found that DNA is precipitated out by adding an organic solvent to a mixed solution of a sample containing DNA and RNA and a chaotropic agent, so that RNA remains soluble. The present invention relates to a method whereby a sample containing nucleic acid, a chaotropic agent, and an organic solvent are mixed, DNA is precipitated out, and the precipitate is separated from the mixed solution, such that RNA is isolated from the residual solution. In addition, in accordance with the present invention, RNA is allowed to come into contact with a silica-containing solid phase so as to be bound to the silica-containing solid phase without the addition of a reagent or the like to the residual solution. Further, it is also possible to isolate DNA from the precipitate.

In accordance with the present invention, high-purity RNA can be isolated from a sample containing DNA and RNA via safe and convenient operations. In addition, it is possible to simultaneously isolate RNA and DNA from a single sample.

### BRIEF DESCRIPTION OF THE DRAWINGS

FIG. 1 shows a schematic diagram of a tip-type instrument for DNA isolation.

FIG. 2 shows a schematic diagram of a column-type instrument for DNA isolation.

FIG. 3 shows a schematic diagram of a tip-type instrument for RNA isolation.

FIG. 4 shows a schematic diagram of a column-type instrument for RNA isolation.

FIG. 5 shows a schematic diagram of an instrument for DNA and RNA isolation.

### DESCRIPTION OF THE PREFERRED EMBODIMENTS

Hereafter, novel features and effects of the present invention described above and other novel features and effects of the present invention are explained by referring to the drawings.

### EXAMPLES

In the following examples, a sample containing nucleic acid, a chaotropic agent, and an organic solvent are mixed and, mainly DNA is precipitated out, such that the precipitate is separated from the mixed solution. Then, the mixed solution from which the precipitate has been separated is allowed to come into contact with a silica-containing solid phase; RNA is allowed to be bound to the silica-containing solid phase; the silica-containing solid phase is separated from the mixed solution; impurities bound to the silica-containing solid phase are removed using a washing reagent; and RNA bound to the silica-containing solid phase is eluted using a elution reagent. In addition, DNA can be purified from the precipitate separated from the mixed solution.

To precipitate out DNA, it is necessary to optimize the organic solvent concentration in the mixed solution after adding a chaotropic agent and an organic solvent to a sample containing nucleic acid. When the organic solvent concentration is below the optimum concentration range, DNA is not precipitated out, while on the other hand, when the organic solvent concentration is above the optimum concentration range, both DNA and RNA are precipitated out. The optimum organic solvent concentration differs mainly depending on type of organic solvent. After the addition of the organic solvent, pipetting or mixing using a mixer or the like promote to precipitate out DNA.

Examples of a sample containing nucleic acid that is a starting material from which RNA or DNA is extracted and isolated include a solution and a biological sample that contain DNA and RNA. As such a solution containing DNA and RNA, for example, a solution obtained by crudely purifying nucleic acid containing both DNA and RNA from a biological sample containing DNA and RNA can be used. In addition, as such a biological sample containing DNA and RNA, whole blood, biomedical tissue, cultured cells, bacteria, or the like can be used.

A chaotropic agent is added to a sample containing nucleic acid so as to help nucleic acid to become bound to a silica-containing solid phase. Examples of such chaotropic agent that can be used include guanidine thiocyanate, sodium thiocyanate, guanidine hydrochloride, sodium iodide, and potassium iodide. Such chaotropic agent can be used at concentrations in the range of 1.0 to 4.0 mol/l in the mixed solution to which an organic solvent has been added. Note that, when a biological sample is subjected to DNA and RNA isolation, a surfactant, a protein denaturant, protease, or the like are added to the sample, in addition to the chaotropic agent. Further, it is preferable to help a biological sample to be lysed and DNA and RNA to be released by allowing the mixed solution to be subjected to physical treatment or the like using a mixer, a homogenizer, or another instrument.

Examples of an organic solvent that can be used include a combination of one or more compounds selected from the group consisting of aliphatic alcohol, aliphatic ether, aliphatic ester, and aliphatic ketone. Examples of aliphatic alcohol that can be used include methanol, ethanol, 2-propanol, 2-butanol, and polyethylene glycol. Examples of aliphatic ether that can be used include diethylene glycol dimethyl ether, diethylene glycol diethyl ether, ethylene glycol dimethyl ether, ethylene glycol diethyl ether, propione glycol dimethyl ether, propylene glycol diethyl ether, and tetrahydrofuran. Examples of aliphatic ester that can be used include ethyl lactate and propylene glycol monomethyl ether acetate. Examples of aliphatic ketone that can be used include acetone, hydroxyacetone, and methylketone.

The precipitate is separated from the mixed solution by a method of filtering the mixed solution or a method of allowing the mixed solution to be subjected to centrifugation. To separate the precipitate from the mixed solution, a member that is equipped with a mixed-solution-introducing opening for introducing a mixed solution of a sample containing nucleic acid, a chaotropic agent, and an organic solvent, a filter for separating precipitated DNA from the mixed solution, and a mixed-solution-discharging opening for discharging the mixed solution that has passed through the filter is used. For instance, filtration of the mixed solution can be carried out by allowing the mixed solution to pass through a column, a tip, or a syringe that accommodates a filter composed of non-silica-containing material, which has a pore size sufficient to entrap the precipitate, and to which RNA in the mixed solution is not bound. Such filter that can be used has pore sizes between 0.1 μm to 500 μm, such that the filter can entrap the precipitate. In addition, as non-silica-containing material to which RNA in a mixed solution is not bound, polypropylene, nylon, polyester, polyvinylidene fluoride, or the like can be used.

The mixed solution from which the precipitate has been separated is allowed to come into contact with a silica-containing solid phase via a method whereby the silica-containing solid phase and the mixed solution are agitated in a vessel so as to be mixed, or via a method whereby the mixed solution is allowed to pass through a member that is equipped with a solution-introducing opening for introducing the mixed solution and a silica-containing solid phase that is disposed so as to be able to come into contact with the mixed solution introduced into the solution-introducing opening. As such member that is equipped with a silica-containing solid phase, for example, a column, a syringe, or a tip to which a silica-containing solid phase is fixed can be used. After allowing the mixed solution to come into contact with a silica-containing solid phase, the mixed solution is separated from the silica-containing solid phase. As a silica-containing solid phase, matter containing silicon oxide in the form of glass particles, silica particles, glass fiber, silica fiber, diatomaceous earth, or disrupted products thereof can be used.

The washing reagent is allowed to come into contact with the silica-containing solid phase via a method whereby the silica-containing solid phase and the washing reagent are agitated in a vessel so as to be mixed, or via a method whereby the washing reagent is allowed to pass through, for example, a column, a tip, or a syringe to which the silica-containing solid phase is fixed. After allowing the washing reagent to come into contact with the silica-containing solid phase, the washing reagent is separated from the silica-containing solid phase. As such washing reagent, an aqueous solution containing ethanol (EtOH) (80% (v/v) or more) or a buffer solution containing EtOH (80% (v/v) or more) with a low salt concentration, which can retain the bond between RNA and the silica-containing solid phase and remove impurities bound to the silica-containing solid phase, may be used.

The elution reagent is allowed to come into contact with the silica-containing solid phase via a method whereby the silica-containing solid phase and the elution reagent are agitated in a vessel so as to be mixed, or via a method whereby the elution reagent is allowed to pass through, for example, a column, a tip, or a syringe to which the silica-containing solid phase is fixed. After allowing the elution reagent to come into contact with the silica-containing solid phase, the elution reagent is separated from the silica-containing solid phase so as to be collected. As such elution reagent, nuclease-free water or a nuclease-free buffer solution with a low salt concentration that can elute RNA binding to the silica-containing solid phase can be used.

DNA isolation from the precipitate is carried out via a method whereby, when the precipitate is separated by filtration of the mixed solution, the washing reagent is allowed to come into contact with the filter that has entrapped the precipitate such that impurities are removed, and then the elution reagent is allowed to come into contact with the filter such that DNA can be eluted. In addition, when the precipitate is separated by centrifugation of the mixed solution, DNA isolation from a precipitate is carried out via a method whereby the obtained supernatant is removed, the washing reagent is added to the precipitate that has been precipitated, impurities contained in the precipitate are precipitated out, the precipitate is precipitated again by centrifugation, the obtained supernatant is removed, and a DNA elution reagent is added to the precipitate that has been precipitated, such that DNA can be eluted. An example of such washing reagent that can be used is an aqueous solution containing EtOH (70% (v/v) or more) or a buffer solution containing EtOH (70% (v/v) or more) with a low salt concentration, which solubilizes impurities contained in the precipitate other than DNA. An example of such elution reagent that can be used is nuclease-free water or a nuclease-free buffer solution with a low salt concentration, which can solubilize DNA. In addition, to further improve purity and yield of DNA isolated from a precipitate, DNA can be reisolated via a method such as ethanol precipitation after the precipitate is lysed in nuclease-free water or a nuclease-free buffer solution with a low salt concentration.

As described above, in examples of the present invention, the composition of a mixed solution that precipitates out DNA is the same as that of a mixed solution that binds RNA with a silica-containing solid phase, so that there is no need to separately establish the condition of DNA separation or the binding condition of RNA with respect to a silica-containing solid phase, or the binding conditions of DNA or RNA with respect to a silica-containing solid phase. Therefore, a mixed solution from which precipitated DNA has been separated is allowed to immediately come into contact with a silica-containing solid phase, such that RNA isolation can be carried out. In a specific example thereof, a mixed solution containing precipitated DNA is allowed to continuously pass through from a filter capable of entrapping precipitated DNA to a silica-containing filter to which RNA can be bound, such that DNA and RNA isolation can be carried out. Thus, the ease of operations for DNA and RNA isolation can be significantly improved.

(Experimentation)

Hereafter, verification experiments of the examples will be described.

A) Materials, Reagents, and Instruments used in the Experiments will be Described Below.

1. Biological Samples Containing DNA and RNA

1.1 White Blood Cells

White blood cells isolated from human whole blood collected in a vacuum tube containing EDTA·2Na as an anticoagulant were used.

1.2 Cultured Cells

Cultured cells of mouse myeloma (So2/0-Ag14) (Dainippon Pharmaceutical) were used.

1.3 Biomedical Tissue

A mouse liver (Funakoshi) was used.

2. Reagents

2.1 A Red Blood Cell Lysis Reagent

155 mM NH₄Cl

10 mM KHCO₃

0.1 mM EDTA·2Na

2.2 A Lysis Reagent

4M GuSCN

25 mM sodium citrate (pH 7.5)

1% β-mercaptoethanol

2.3 Organic Solvent Solutions

(1) 40% (v/v) diethylene glycol dimethyl ether aqueous solution
(2) 42.5% (v/v) diethylene glycol dimethyl ether aqueous solution
(3) 45% (v/v) diethylene glycol dimethyl ether aqueous solution
(4) 47.5% (v/v) diethylene glycol dimethyl ether aqueous solution
(5) 50% (v/v) diethylene glycol dimethyl ether aqueous solution
(6) 65% (v/v) 2-propanol aqueous solution
(7) 67.5% (v/v) 2-propanol aqueous solution
(8) 70% (v/v) 2-propanol aqueous solution
(9) 72.5% (v/v) 2-propanol aqueous solution
(10) 75% (v/v) 2-propanol aqueous solution
(11) 77.5% (v/v) ethanol aqueous solution
(12) 70% (v/v) 2-butanol aqueous solution
(13) 50% (v/v) polyethylene glycol (average molecular weight of 300) aqueous solution
(14) 70% (v/v) ethyl lactate aqueous solution
   2.4 Washing Reagents
   (1) A DNA Washing Reagent

80% (v/v) ethanol aqueous solution

(2) An RNA Washing Reagent

80% (v/v) ethanol aqueous solution

2.5 Elution Reagents

(1) A DNA Elution Reagent

TE (pH 8.0) (Wako Pure Chemical)

(2) An RNA Elution Reagent

H₂O (nuclease-free) (Wako Pure Chemical)

3. An Instrument for DNA Isolation

(1) A Filter for Precipitate Separation

A sintered plate of polypropylene particles having a pore size of 100 μm (2 mm thick) was used.

(2) A Tip-Type Instrument for DNA Isolation

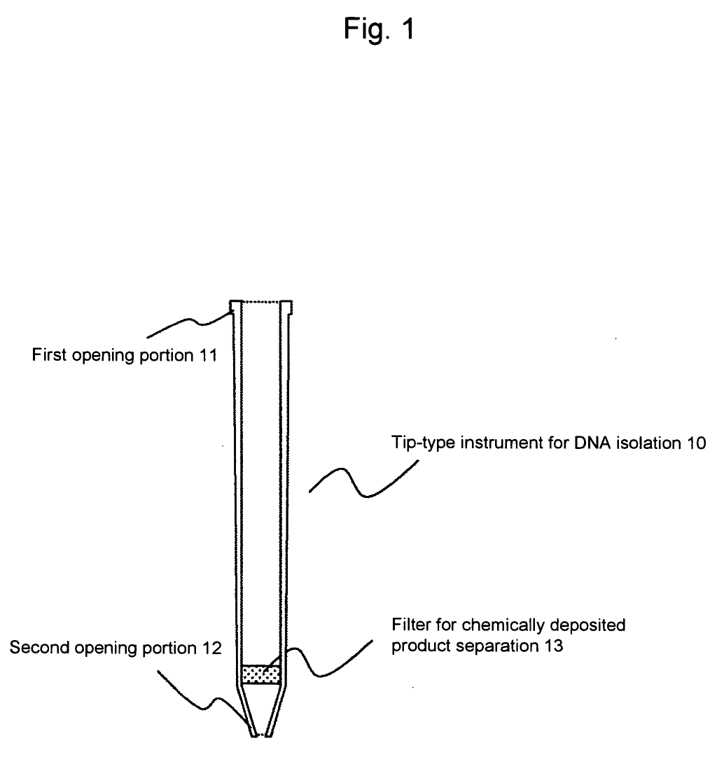

FIG. 1 shows a constitutional example of a tip-type instrument for DNA isolation. A tip-type instrument for RNA isolation 10 has an appearance like a tip dispenser. The instrument is equipped with a first opening portion 11 through which a solution can be introduced and a second opening portion 12 from which a solution can be discharged, and accommodates a filter for precipitate separation 13 inside itself. When a first opening portion of the instrument is attached to a pressure control system, a solution can be aspirated and discharged via a second opening portion. During experimentation, the filter for precipitate separation was used after being cut into a cylindrical shape with a diameter of 4.2 mm so as to be press-fitted into a tip 4 mm in inner diameter.

(3) A Spin-Column-Type Instrument for DNA Isolation

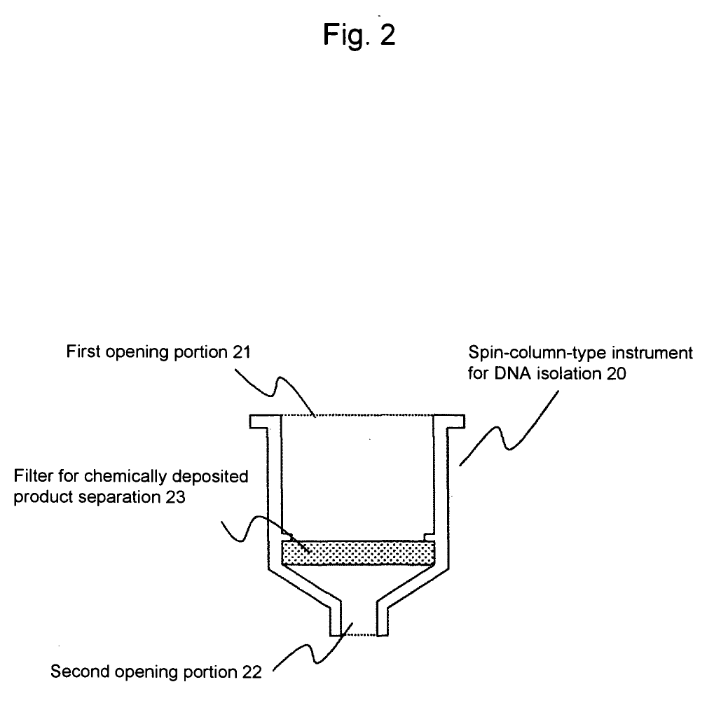

FIG. 2 shows a constitutional example of a spin-column-type instrument for DNA isolation. A spin-column-type instrument for DNA isolation 20 has an appearance like a spin column. The instrument is equipped with a first opening portion 21 through which a solution can be introduced and a second opening portion 22 from which a solution can be discharged, and accommodates a filter for precipitate separation 23 inside itself. When the instrument is mounted on a centrifugal separator, the solution introduced is allowed to pass through the filter for precipitate separation by centrifugation so as to be discharged. During experimentation, a filter for precipitate separation was used after being cut into a disc shape with a diameter of 7.6 mm so as to be press-fitted into a spin column 7.5 mm in inner diameter.

4. An Instrument for RNA Isolation

(1) A Silica-Containing Solid Phase

A glass fiber filter (GF/D, Whatman) was used.

(2) A Silica-Containing Solid-Phase-Retaining Member

A sintered plate of polypropylene particles having a pore size of 100 μm (1.5 mm thick) was used.

(3) A Tip-Type Instrument for RNA Isolation

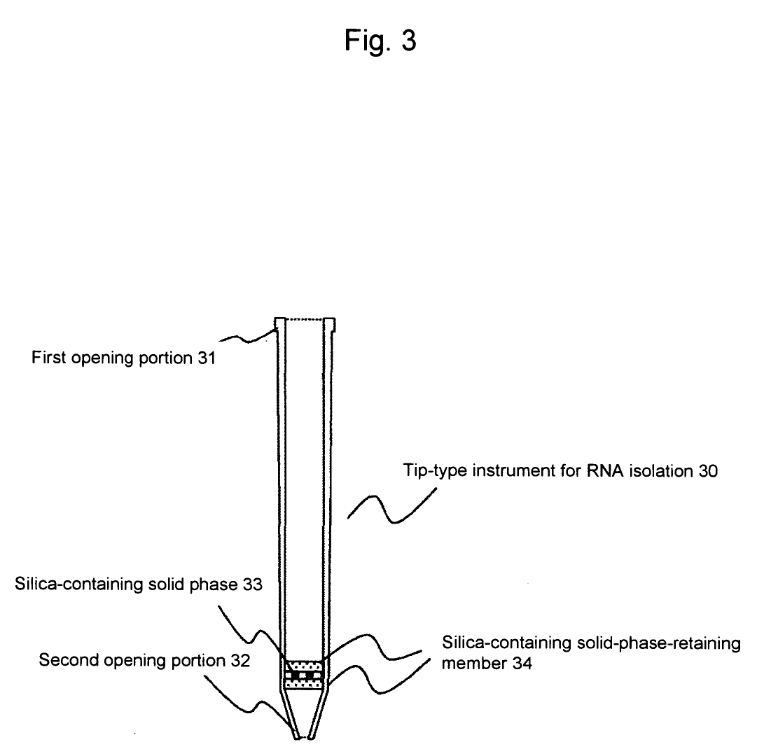

FIG. 3 shows a constitutional example of a tip-type instrument for RNA isolation. The tip-type instrument for RNA isolation 30 has an appearance like a tip dispenser. The instrument is equipped with a first opening portion 31 that can be attached to a pressure control system and a second opening portion 32 from which a solution can be aspirated and discharged, and accommodates a silica-containing solid phase 33 inside itself. On each side of the silica-containing solid phase, a disc-shaped silica-containing solid-phase-retaining member 34 is disposed. These silica-containing solid-phase-retaining members have a number of pores that have been formed therein, through which liquid and gas freely pass. When a first opening portion of the instrument is attached to a pressure control system, a solution can be aspirated and discharged via a second opening portion. During experimentation, a silica-containing solid phase that has been cut into a disc shape with a diameter of 4.2 mm was used while being sandwiched by two silica-containing solid-phase-retaining members that had each been cut into a disc shape with a diameter of 4.1 mm so as to be press-fitted into a hollow tip 4 mm in inner diameter.

(4) A Spin-Column-Type Instrument for RNA Isolation

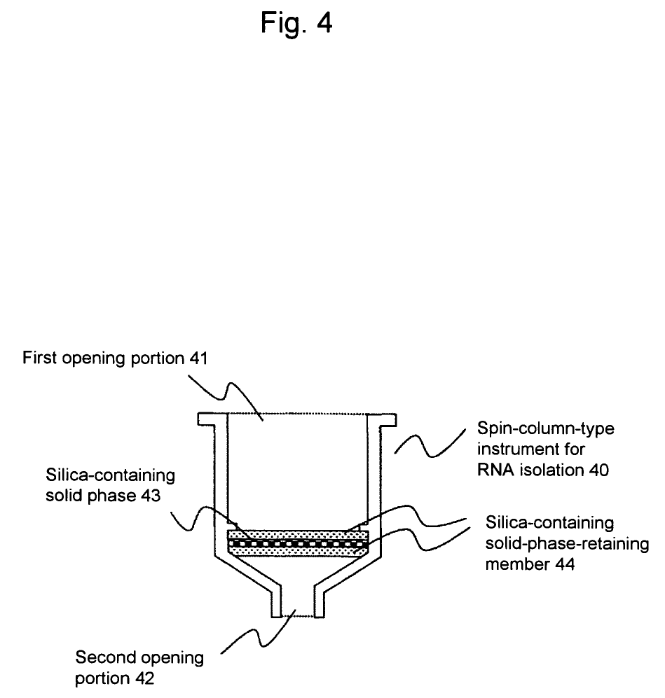

FIG. 4 shows a constitutional example of a spin-column-type instrument for RNA isolation. A spin-column-type instrument for RNA isolation 40 has an appearance like a spin column. The instrument is equipped with a first opening portion 41 through which solution can be introduced and a second opening portion 42 from which solution can be discharged, and accommodates a silica-containing phase 43 inside thereof, at each end of which a silica-containing phase-retaining member 44 is disposed. When the instrument is mounted on a centrifugal separator, the solution introduced is allowed to pass through the silica-containing phase so as to be discharged by centrifugation. During the experimentation, two silica-containing solid phases that had each been cut into a disc shape with a diameter of 7.7 mm were used while being sandwiched by two silica-containing solid phase members that had each been cut into a disc shape with a diameter of 7.6 mm so as to be press-fitted into a spin column 7.5 mm in inner diameter.

5. An Instrument for DNA and RNA Isolation

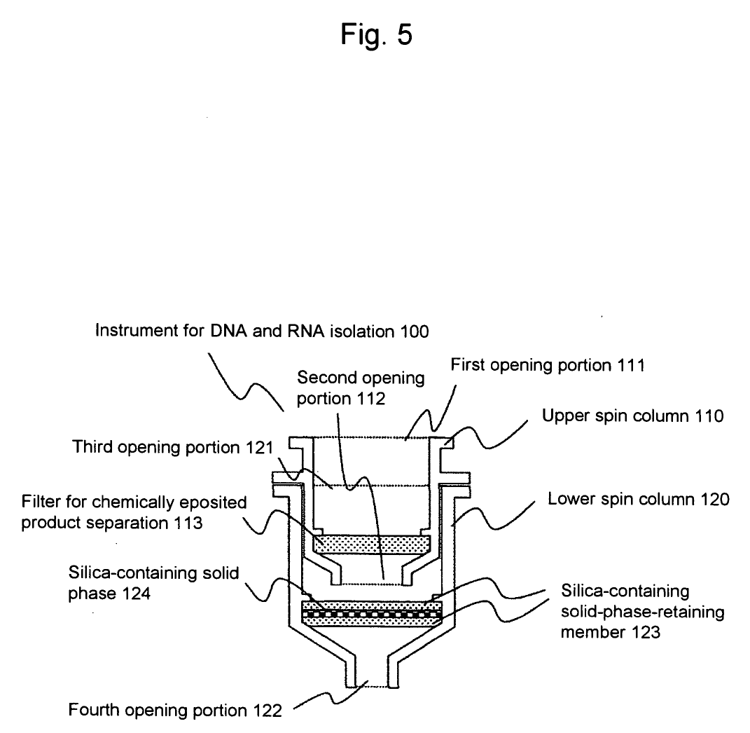

FIG. 5 shows a constitutional example of an instrument for DNA and RNA isolation. An instrument for DNA and RNA isolation 100 is a combination of a spin-column-type instrument for DNA isolation and a spin-column-type instrument for RNA isolation. The instrument is composed of an upper spin column 110 as an instrument for DNA isolation and a lower spin column 120 as an instrument for RNA isolation. The upper spin column 110 is equipped with a first opening portion 111 through which a solution can be introduced and a second opening portion 112 from which a solution can be discharged, and accommodates a filter for precipitate separation 113 inside itself. The lower spin column 120 is connected with the upper spin column, is equipped with a third opening portion 121 through which the solution discharged from the upper spin column can be introduced and a fourth opening portion 122 from which the solution can be discharged, and accommodates a silica-containing solid phase 124 inside itself. At each end of such phase, a silica-containing solid-phase-retaining member 123 is disposed. When the instrument is mounted on a centrifugal separator, the solution introduced is allowed to continuously pass through the filter for precipitate separation and the silica-containing solid phase by centrifugation so as to be discharged. During the experimentation, a two-tiered instrument comprised of the upper spin column and the lower spin column was used while a filter for precipitate separation that had been cut into a disc shape with a diameter of 6.8 mm was press-fitted into the upper spin column 6.7 mm in inner diameter and two sheets of glass fiber filter paper that had each been cut into a cylindrical shape with a diameter of 7.7 mm are sandwiched by two silica-containing solid-phase-retaining members that had each been cut into a disc shape with a diameter of 7.6 mm so as to be press-fitted into the lower spin column 7.5 mm in inner diameter.

B) Individual Methods used in Experimentation will be Described Below.

A method for DNA and RNA isolation from a biological sample containing DNA and RNA may be carried out as a combination of the individual methods described below according to need.

1. A Method for Isolating White Blood Cells from Whole Blood

(1) A red blood cell lysis reagent is added to whole blood in a volume 5 times that of the total volume of whole blood, followed by mixing.
(2) The mixture is incubated on ice for 5 minutes.
(3) Centrifugation is performed at 400×g for 10 minutes.
(4) The supernatant of the resultant was removed.
(5) A red blood cell lysis reagent is added to the pellet obtained in a volume 2 times that of the reagent added to the whole blood, followed by mixing.
(6) Centrifugation is performed at 400×g for 10 minutes under the 4° C. environment.
(7) The supernatant of the resultant is removed such that a white blood cell pellet can be obtained.
   2. A Method for Dissolving a Biological Sample
   2.1 A Method for Dissolving White Blood Cells
(1) A lysis reagent is added to a white blood cell pellet in a volume that is half of the total volume of the whole blood from which white blood cells have been isolated, followed by mixing.
(2) The mixed solution is homogenized using a homogenizer (QIA shredder homogenizer; Qiagen).
   2.2 A Method for Dissolving Cultured Cells
(1) A lysis reagent (1 ml) is added to a cell pellet containing 5×10⁵ cells, followed by mixing.
(2) The mixed solution is homogenized using a homogenizer (T 8 Ultra-Turrax; Ika).
   2.3 A Method for Dissolving Biomedical Tissue
(1) A lysis reagent (1 ml) is added to 1 mg of biomedical tissue, followed by mixing.
(2) The mixed solution is homogenized using a homogenizer (T 8 Ultra-Turrax; Ika).
   3. A Method for Precipitating DNA

An organic solvent is added to the homogenized mixed solution in a volume that is the same as that of the lysis reagent added to the biological sample, followed by sufficient mixing.

4. A Method for DNA Isolation

4.1 A Method for DNA Isolation using a Tip-Type Instrument for DNA Isolation

(1) A mixed solution in which a precipitate has been formed is added into a tip from the upper part of the tip.
(2) A syringe is attached to the tip such that the solution inside the tip is discharged from the lower part of the chip.
(3) The syringe is disconnected from the tip and a DNA washing reagent is added into the tip from the upper part of the tip in an amount equal to that of the mixed solution.
(4) The syringe is attached to the tip such that the DNA washing reagent inside the tip is discharged from the lower part of the chip.
(5) Steps (3) and (4) are repeated twice.
(6) A DNA elution reagent is added to a vessel for purified products in a volume that is ⅕ that of the mixed solution.
(7) The DNA elution reagent is aspirated from the lower part of the tip until the reagent has passed through the filter for precipitate separation so as to be incubated at room temperature for 3 minutes.
(8) The DNA elution reagent is discharged from the lower part of the tip to the vessel for purified products.
(9) The DNA elution reagent is aspirated from the lower part of the tip until the reagent has passed through the filter for precipitate separation so as to be discharged from the lower part of the tip to the vessel for purified products.
(10) Step (9) is repeated 9 times such that a DNA isolation solution can be obtained in the vessel for purified products.
   4.2 A Method for DNA Isolation using a Spin-Column-Type Instrument for DNA Isolation
(1) A mixed solution in which a precipitate has been formed is added into a spin column.
(2) A solution-receiving vessel is installed under the spin column, centrifugation is performed at 2000×g for 10 seconds, and then the solution inside the spin column is discharged into the solution-receiving vessel.
(3) A DNA washing reagent is added into the spin column in an amount equal to that of the mixed solution.
(4) The solution-receiving vessel is installed under the spin column, centrifugation is performed at 2000×g for 10 seconds, and then the solution inside the spin column is discharged into the solution-receiving vessel.
(5) Steps (3) and (4) are repeated twice.
(6) A DNA elution reagent is added into the spin column in a volume that is ⅕ that of the mixed solution.
(7) Incubation is performed at 60° C. for 3 minutes.
(8) A solution-receiving vessel for purified products is installed under the spin column, centrifugation is performed at 2000×g for 1 minute, and then the solution inside the spin column is discharged into the solution-receiving vessel for purified products, such that a DNA isolation solution can be obtained.
   4.3 A Method for Isolating and Purifying DNA by Centrifugation
(1) A mixed solution in which a precipitate has been formed is subjected to centrifugation at 6000×g for 3 minutes.
(2) The thus obtained supernatant is transferred to a different vessel.
(3) A DNA washing reagent is added to the obtained precipitate in an amount equal to that of the mixed solution, followed by mixing.
(4) Centrifugation is performed at 10000×g for 5 minutes.
(5) The thus obtained supernatant is discarded, and a DNA washing reagent is added to the obtained precipitate in an amount equal to that of the mixed solution, followed by mixing.
(6) Centrifugation is performed at 10000×g for 5 minutes.
(7) The thus obtained supernatant is discarded, and a DNA washing reagent is added to the obtained precipitate in a volume that is ⅕ that of the mixed solution, followed by mixing, such that a DNA isolation solution can be obtained.
   5. A Method for RNA Isolation
   5.1 A Method for RNA Isolation using a Tip-Type Instrument for RNA Isolation
(1) A syringe is attached to a chip.
(2) A mixed solution that has passed through a filter for precipitate separation is aspirated from the lower part of the tip until the reagent has passed through a silica-containing solid phase so as to be discharged to a vessel.
(3) Step (2) is repeated 5 times.
(4) The syringe is disconnected from the tip and an RNA washing reagent is added into the tip from the upper part of the tip in an amount equal to that of the mixed solution.
(5) The syringe is attached to the tip such that the RNA washing reagent inside the tip is discharged from the lower part of the chip.
(6) Steps (4) and (5) are repeated twice.
(7) An RNA elution reagent is added to a vessel for purified products in a volume that is 1/20 that of the mixed solution.
(8) An RNA elution reagent is aspirated from the lower part of the tip until the reagent has passed through the silica-containing solid phase so as to be discharged from the lower part of the tip to the vessel for purified products.
(9) Step (8) is repeated 10 times such that an RNA isolation solution can be obtained in the vessel for purified products.
   5.2 A Method for RNA Isolation using a Spin-Column-Type Instrument for RNA Isolation
(1) A mixed solution that has passed through a filter for precipitate separation is added into a spin column.
(2) A solution-receiving vessel is installed under the spin column, centrifugation is performed at 4000×g for 1 minute, and then the solution inside the spin column is discharged into the solution-receiving vessel.
(3) An RNA washing reagent is added into the spin column in an amount equal to that of the mixed solution.
(4) A solution-receiving vessel is installed under the spin column, centrifugation is performed at 4000×g for 1 minute, and then the solution inside the spin column is discharged into the solution-receiving vessel.
(5) Steps (3) and (4) are repeated twice.
(6) An RNA elution reagent is added into the spin column in a volume that is 1/20 that of the mixed solution.
(7) A solution-receiving vessel for purified products is installed under the spin column, centrifugation is performed at 4000×g for 1 minute, and then the solution inside the spin column is discharged into the solution-receiving vessel for purified products, such that an RNA isolation solution can be obtained.
   6. A Method for DNA and RNA Isolation using an Instrument for DNA and RNA Isolation
(1) A mixed solution in which a precipitate has been formed is added into the upper spin column of an instrument for DNA and RNA isolation.
(2) A solution-receiving vessel is installed under the spin column, centrifugation is performed at 4000×g for 1 minute, and then the solution inside the spin column is discharged into the solution-receiving vessel.
(3) The upper spin column and the lower spin column of the instrument for DNA and RNA continuous isolation are separated.
(4) A DNA washing reagent is added into the upper spin column in an amount equal to that of the mixed solution.
(5) An RNA washing reagent is added into the lower spin column in an amount equal to that of the mixed solution.
(6) A solution-receiving vessel is installed under each spin column, centrifugation is performed at 4000×g for 1 minute, and then the solution inside each spin column is discharged into each corresponding solution-receiving vessel.
(7) Steps (3) and (4) are repeated twice.
(8) A DNA elution reagent is added into the upper spin column in a volume that is ⅕ that of the mixed solution, followed by incubation at 65° C. for 3 minutes.
(9) An RNA elution reagent is added into the lower spin column in a volume that is 1/20 that of the mixed solution.
(10) A solution-receiving vessel for purified products is installed under each spin column, centrifugation is performed at 4000×g for 1 minute, and then the solution inside each spin column is discharged into each corresponding solution-receiving vessel for purified products, such that a DNA isolation solution and an RNA isolation solution can be separately obtained.
   C) Hereafter, a Method for Evaluating DNA and RNA Isolated in Experimentation will be Described.
   1. Calculation of DNA and RNA Content Ratios by Electrophoresis

Using a 1.25% agarose gel (Reliant RNA Gel System, FMC), electrophoresis (10 V/cm, 40 minutes) was performed on a DNA isolation solution and an RNA isolation solution that had been subjected to formamide denaturation. After electrophoresis, agarose gel was dyed with ethidium bromide. Then, the fluorescent intensity of an RNA group containing mRNA and rRNA and the fluorescent intensity of genomic DNA were determined using a densitograph (Atto) under UV irradiation. Thus, DNA and RNA content ratios were calculated based on the fluorescent intensity ratio of RNA to DNA.

2. Quantification of DNA and RNA Concentrations

A DNA isolation solution and an RNA isolation solution were adequately diluted such that absorbance of each solution at 260 nm was determined using a spectrophotometer (GeneSpecI, Hitachi Naka Instrument). Then amounts of DNA and RNA were calculated, where concentration factors of DNA and RNA were 50 μg/ml and 40 μg/ml, respectively, based on the DNA and RNA content ratios calculated by densitograph analysis.

D) Verification Experiment 1

It is necessary to optimize the concentration of an organic solvent so as to achieve DNA and RNA isolation by adding the organic solvent to a mixed solution of a biological sample containing DNA and RNA and a chaotropic agent, such that DNA is precipitated out. In this experiment, to evaluate the relationship between effects of DNA and RNA isolation and organic solvent concentrations, optimization of organic solvent concentration was examined by changing the concentration of an organic solvent using diethylene glycol dimethyl ether and 2-propanol as organic solvents such that DNA and RNA were isolated.
Biological sample: white blood cells (equal to 600 μl of whole blood)
Organic solvents: a diethylene glycol dimethyl ether aqueous solution and a 2-propanol aqueous solution
Method of DNA isolation: a method of DNA isolation using a tip-type instrument for DNA isolation
Method of RNA isolation: a method of RNA isolation using a spin-column-type instrument for RNA isolation

Table 1 below shows DNA and RNA contents of a DNA isolation solution and RNA and DNA contents of an RNA isolation solution.

When using diethylene glycol dimethyl ether at a concentration of about 45%, the DNA isolation solution scarcely contained RNA and the RNA isolation solution scarcely contained DNA. Therefore, DNA and RNA were isolated at high purity. Meanwhile, when using diethylene glycol dimethyl ether at a concentration of 40%, the DNA content in the DNA isolation solution tended to decrease; however, the DNA content in the RNA isolation solution tended to increase. In addition, when using diethylene glycol dimethyl ether at a concentration of 50%, the RNA content in the DNA isolation solution tended to increase; however, the RNA content in the RNA isolation solution tended to decrease.

When using 2-propanol at a concentration of about 70%, the DNA isolation solution scarcely contained RNA and the RNA isolation solution scarcely contained DNA. Therefore, DNA and RNA were isolated at high purity. Meanwhile, when using diethylene glycol dimethyl ether at concentration of a 65%, the DNA content in the DNA isolation solution tended to decrease; however, the DNA content in the RNA isolation solution tended to increase. In addition, when using diethylene glycol dimethyl ether at a concentration of 75%, the RNA content in the DNA isolation solution tended to increase; however, the RNA content in the RNA isolation solution tended to decrease.

Fluctuation of the RNA and DNA content ratios depending on organic solvent concentration is considered to be due to the following reason: when the organic solvent concentration is below the optimum concentration range, DNA is not precipitated out completely so that soluble DNA is bound to a silica-containing solid phase together with RNA; when the organic solvent concentration is above the optimum concentration range, both DNA and RNA are precipitated out so that precipitated RNA is caught by a filter for precipitate separation together with DNA.

[Table 1]

TABLE 1

| | | DNA isolation | RNA isolation |
|---|---|---|---|
| | | solution | solution |
| Organic | Concentration | DNA content (μg) | RNA content (μg) |
| solvent | % (v/v) | (RNA content (μg)) | (DNA content (μg)) |
| Diethylene | 40 | 8.9 | 2.8 |
| glycol | | (0.0) | (3.2) |
| dimethyl | 42.5 | 14.8 | 2.6 |
| ether | | (0.0) | (0.2) |
| | 45 | 15.2 | 2.5 |
| | | (0.0) | (0.1) |
| | 47.5 | 15.4 | 2.1 |
| | | (0.3) | (0.0) |
| | 50 | 15.9 | 0.8 |
| | | (0.5) | (0.0) |
| 2-propanol | 65 | 4.1 | 2.9 |
| | | (0.0) | (4.7) |
| | 67.5 | 15.4 | 2.7 |
| | | (0.0) | (0.2) |
| | 70 | 15.5 | 2.4 |
| | | (0.0) | (0.1) |
| | 72.5 | 15.3 | 2.2 |
| | | (0.3) | (0.0) |
| | 75 | 16.1 | 1.3 |
| | | (0.5) | (0.0) |

E) Verification Experiment 2

Various types of organic solvents can be used for carrying out a method for DNA and RNA isolation wherein an organic solvent is added to a mixed solution of a biological sample containing DNA and RNA and a chaotropic agent such that only DNA is precipitated out. In this experiment, to evaluate the relationship between effects of DNA and RNA isolation and types of organic solvent, DNA and RNA isolation was carried out at the optimum concentration determined based on the method of verification examination 1 using ethanol, 2-propanol, 2-butanol, polyethylene glycol, ethyl lactate, and diethylene glycol dimethyl ether as organic solvents.
Biological sample: white blood cells (equal to 600 μl of whole blood)
Method for DNA isolation: a method for DNA isolation using a tip-type instrument for DNA isolation
Method for RNA isolation: a method for RNA isolation using a spin-column-type instrument for RNA isolation

Table 2 below shows DNA and RNA contents of a DNA isolation solution and RNA and DNA contents of an RNA isolation solution. At a given concentration of each organic solvent, the DNA isolation solution scarcely contained RNA and the RNA isolation solution scarcely contained DNA. Therefore, DNA and RNA were isolated at high purity.

[Table 2]

TABLE 2

| | | DNA isolation | RNA isolation |
|---|---|---|---|
| | | solution | solution |
| | | DNA content (μg) | RNA content (μg) |
| | Concentration | (RNA content | (DNA content |
| Organic solvent | % (v/v) | (μg)) | (μg)) |
| ethanol | 77.5 | 13.5 | 1.6 |
| | | (0.0) | (0.2) |
| 2-propanol | 70 | 15.5 | 2.4 |
| | | (0.0) | (0.1) |
| 2-butanol | 70 | 12.8 | 2.0 |
| | | (0.1) | (0.1) |
| polyethylene | 50 | 13.7 | 1.8 |
| glycol | | (0.1) | (0.1) |
| ethyl lactate | 70 | 14.9 | 2.5 |
| | | (0.0) | (0.1) |
| Diethylene glycol | 45 | 15.2 | 2.5 |
| dimethyl ether | | (0.0) | (0.1) |

F) Verification Experiment 3

Various types of instruments for isolation can be used for carrying out a method for DNA and RNA isolation wherein an organic solvent is added to a mixed solution of a biological sample containing DNA and RNA and a chaotropic agent such that only DNA is precipitated out. In this experiment, to evaluate the relationship between effects of DNA and RNA isolation and types of instrument for isolation, DNA and RNA isolation was carried out by a method using an instrument for DNA isolation and an instrument for RNA isolation, which were spin-column-type or tip-type, under the following conditions.
Biological sample: white blood cells (equal to 600 μl of whole blood)
Organic solvent: a 70% (v/v) 2-propanol aqueous solution

Table 3 below shows DNA contents and RNA content ratios of a DNA isolation solution, and RNA contents and DNA content ratios of an RNA isolation solution. With each method, the DNA isolation solution scarcely contained RNA and the RNA isolation solution scarcely contained DNA. Therefore, DNA and RNA were isolated at high purity.

The largest content of DNA in a DNA isolation solution was obtained via a method of centrifugation, the second largest was obtained via a method using a tip-type instrument for isolation, and the third largest was obtained via a method using a spin-column-type instrument for isolation. It is considered that this is because the elution efficiency of DNA differs in each method, although the entrapment efficiency of precipitated DNA is almost the same in each method. In addition, the amount of RNA in the RNA isolation solution obtained via the method using a tip-type instrument for isolation was larger than that obtained via the method using a spin-column-type instrument for isolation. It is considered that this is because the binding efficiency and the elution efficiency of RNA differ in each method.

Meanwhile, the shortest period of time necessary for operation of DNA and RNA isolation was in the case of the method using an instrument for DNA and RNA isolation, the second shortest was in the case of the method using a spin-column-type instrument for isolation, and the third shortest was in the case of the method using a tip-type instrument for isolation. This is because the method using a column-type instrument for isolation is more convenient than the method for using a tip-type instrument for isolation in terms of the number of processes for the operation.

In addition, the method for using an instrument for DNA and RNA isolation that is most convenient in terms of operation for DNA and RNA isolation is practicable since the composition of a mixed solution to precipitate DNA is the same as that of a mixed solution for allowing RNA to be bound to a silica-containing solid phase.

[Table 3]

TABLE 3

| | | DNA isolation | RNA isolation |
|---|---|---|---|
| | | solution | solution |
| Instrument for | Instrument for | DNA content (μg) | RNA content (μg) |
| DNA isolation | RNA isolation | (RNA content (μg)) | (DNA content (μg)) |
| Tip-type instrument | Tip-type instrument | 14.0 | 2.7 |
| for isolation | for isolation | (0.1) | (0.1) |
| Tip-type instrument | Spin-column-type | 14.2 | 2.4 |
| for isolation | instrument for | (0.1) | (0.1) |
| | isolation | | |
| Spin-column-type | Tip-type instrument | 11.2 | 2.7 |
| instrument for | for isolation | (0.1) | (0.1) |
| isolation | | | |
| Centrifugal | Tip-type instrument | 16.3 | 2.6 |
| separator | for isolation | (0.2) | (0.1) |

| | | |
|---|---|---|
| Instrument for DNA and RNA isolation | 10.5 | 2.3 |
| | (0.1) | (0.1) |

G) Verification Experiment 4

A method of DNA and RNA isolation wherein an organic solvent is added to a mixed solution of a biological sample containing DNA and RNA and a chaotropic agent such that only DNA is precipitated out can be applied to various types of samples containing nucleic acid. In this experiment, to evaluate the relationship between effects of DNA and RNA isolation and samples containing nucleic acid, DNA and RNA isolation was carried out by a method using various types of biological samples under the following conditions.

Biological Sample:
(1) white blood cells (equal to 600 μl of whole blood)
(2) cultured cells (5×10⁵ cells)
(3) biomedical tissue (1 mg)
Organic solvent: a 70% (v/v) ethyl lactate aqueous solution
Method of DNA isolation: a method of DNA isolation using a tip-type instrument for DNA isolation
Method of RNA isolation: a method of RNA isolation using a tip-type instrument for RNA isolation

Table 4 below shows DNA contents and RNA content ratios of the DNA isolation solution and RNA contents and DNA content ratios of the RNA isolation solution. In various types of biological samples, the DNA isolation solution scarcely contained RNA and the RNA isolation solution scarcely contained DNA. Therefore, DNA and RNA were isolated at high purity.

[Table 4]

TABLE 4

| | DNA isolation solution | RNA isolation solution |
|---|---|---|
| | DNA content (μg) | RNA content (μg) |
| Biological sample | (RNA content (μg)) | (DNA content (μg)) |
| White blood cells | 14.7 | 2.7 |
| | (0.0) | (0.1) |
| Cultured cells | 2.9 | 4.0 |
| | (0.0) | (0.0) |
| Biomedical tissue | 1.1 | 3.7 |
| | (0.0) | (0.0) |

What is claimed is:

1. A method for nucleic acid isolation, comprising steps of:
mixing a sample containing nucleic acid, a chaotropic agent, and an organic solvent to precipitate out DNA;
removing the precipitate from the mixed solution;
allowing the mixed solution from which the precipitate has been separated to come into contact with a silica-containing solid phase such that RNA is allowed to be bound to the silica-containing solid phase;
separating the silica-containing solid phase from the mixed solution;
washing the silica-containing solid phase; and
eluting RNA from the silica-containing solid phase.

2. The method for nucleic acid isolation according to claim 1, wherein the organic solvent is an aliphatic alcohol, aliphatic ether, aliphatic ester, or aliphatic ketone compound, or a combination of any thereof.

3. The method for nucleic acid isolation according to claim 1, wherein the organic solvent is ethanol, 2-propanol, 2-butanol, polyethylene glycol, diethylene glycol dimethyl ether, or ethyl lactate.

4. A method for nucleic acid isolation, comprising steps of:
mixing a sample containing nucleic acid, a chaotropic agent, and an organic solvent to precipitate out DNA;
separating the precipitate from the mixed solution and isolating DNA from the precipitate;
allowing the mixed solution from which the precipitate has been separated to come into contact with a silica-containing solid phase such that RNA is allowed to be bound to the silica-containing solid phase;
separating the silica-containing solid phase from the mixed solution;
washing the silica-containing solid phase; and
eluting RNA from the silica-containing solid phase.

5. The method for nucleic acid isolation according to claim 4, wherein the organic solvent is an aliphatic alcohol, aliphatic ether, aliphatic ester, or aliphatic ketone compound, or a combination of any thereof.

6. The method for nucleic acid isolation according to claim 4, wherein the organic solvent is ethanol, 2-propanol, 2-butanol, polyethylene glycol, diethylene glycol dimethyl ether, or ethyl lactate.

7. An instrument for nucleic acid isolation comprising the following structure:
a separating member that is equipped with a mixed-solution-introducing opening for introducing a mixed solution of a sample containing nucleic acid, a chaotropic agent, and an organic solvent, a filter capable of separating a precipitate containing DNA from the mixed solution, and a mixed-solution-discharging opening for discharging the mixed solution that has passed through the filter; and
a member for nucleic acid entrapment that is equipped with a solution-introducing opening that can be connected with the mixed-solution-discharging opening of the separating member and a silica-containing solid phase that is disposed so as to be able to come into contact with the solution introduced from the solution-introducing opening.

8. The instrument for nucleic acid isolation according to claim 7, in which the mixed solution is allowed to pass through the filter via centrifugal force so as to come into contact with the silica-containing solid phase.

9. The instrument for nucleic acid isolation according to claim 7, in which the mixed solution is allowed to pass through the filter via pressure difference so as to come into contact with a silica-containing solid phase.
